(19) United States
(12) Patent Application Publication
(10) Pub. No.: US 2009/0157089 A1
(43) Pub. Date: Jun. 18, 2009

(54) APPARATUS FOR LOADING AN IMPLANTABLE DEVICE

(75) Inventor: Isaac Plans, Galway (IE)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 11/959,297

(22) Filed: Dec. 18, 2007

(51) Int. Cl. A61F 2/04

(52) U.S. Cl. 606/108

(57) ABSTRACT

Apparatus and methods for loading an implantable device are disclosed. The apparatus for loading an implantable device includes a support portion. The support portion is configured to receive the implantable device. The apparatus includes a stop portion. The stop portion is configured to limit longitudinal motion of the implantable device in at least one direction. The method for loading an implantable device includes positioning an implantable device within a loading apparatus, inserting an implantable device carrying apparatus into the implantable device, and removing the implantable device carrying apparatus and the implantable device from the loading apparatus.

### FIELD OF THE INVENTION

The present invention relates generally to medical devices, and more particular to apparatus for loading an implantable device.

### BACKGROUND OF THE INVENTION

Implantable devices may be used in many medical procedures. For example, balloon angioplasty, either alone or followed by an endoprosthetic implantation, has become a commonplace interventional alternative to open heart surgery in those patients appropriate for such treatment. Endoprostheses are a type of implantable device that include generally tubular members having a collapsed state suitable for insertion into a vessel and a deployed state in which the endoprosthesis is expanded to support the surrounding tissue and prevent at least local narrowing of the vessel. Several types of endoprostheses are known, including balloon expandable, self-expanding, and endoprostheses constructed from biostable springs.

Porous materials, for example, are commonly used in implantable devices as matrices for the retention of therapeutic agents. These materials are typically applied as coatings to the medical devices, raising issues regarding coating adhesion, mechanical properties, cracking, delamination, and material biocompatibility. However, problems may occur when mechanical forces are applied on an endoprosthesis during manufacture (crimping, endoprosthetic retention procedures, packaging etc.) as well as during actual use (unsheathing, catheter preparation, advancement through catheter, vasculature and vascular lesions), which may result in damaging the coating. Specifically, implantable devices and/or their coatings may be damaged when loaded onto an implantable device carrying apparatus.

Accordingly, it may be desirable to provide apparatus and methods for loading an implantable device.

### BRIEF SUMMARY

Described is an apparatus for loading an implantable device. The apparatus includes a support portion configured to receive the implantable device and a stop portion configured to limit longitudinal motion of the implantable device in at least one direction.

A method for loading an implantable device is described. The method includes positioning an implantable device within a loading apparatus. The loading apparatus includes a support portion configured to receive the implantable device. The support portion is configured to impede lateral motion of the implantable device in at least one direction. The loading apparatus includes a stop portion configured to limit longitudinal motion of the implantable device in at least one direction. An implantable device carrying apparatus is inserted into the implantable device. The implantable device carrying apparatus and the implantable device are removed from the loading apparatus. In some embodiments, multiple implantable devices are positioned within the loading apparatus.

A second embodiment of an apparatus for loading an implantable device is described. The apparatus includes a support portion configured to receive the implantable device. The support portion includes at least one support surface. The apparatus includes a stop portion configured to limit longitudinal motion of the implantable device in at least one direction. The stop portion includes a passage configured to allow access to a lumen of the implantable device.

A third embodiment of an apparatus for loading an implantable device is described. The apparatus includes a support portion configured to receive the implantable device. The support portion includes at least one support surface. The at least one support surface is configured to impede lateral motion of the implantable device in at least one direction. The apparatus includes a stop portion configured to limit longitudinal motion of the implantable device in at least one direction. The stop portion includes a passage configured to allow access to a lumen of the implantable device. The support portion is sloped relative to said stop portion from a proximal end of said support portion toward a distal end of said support portion.

In some embodiments, the support portion includes at least one support surface. In further embodiments, the at least one support surface is configured to impede lateral motion of the implantable device in at least one direction. In still further embodiments, a first support surface and a second support surface intersect in an acute angle. The support portion, in yet further embodiments is sloped relative to the stop portion from a proximal end of the support portion toward a distal end of the support portion.

The at least one support surface, in some embodiments, is concave. In further embodiments, the at least one support surface is curved. The stop portion, in still further embodiments, includes a passage configured to allow access to a lumen of the implantable device.

It is to be understood that both the foregoing general description and the following detailed description are exemplary and are intended to provide further explanation of the invention claimed.

The accompanying Figures, which are incorporated in and constitute part of this specification, are included to illustrate and provide a further understanding of the method and system of the invention. Together with the description, the Figures serve to explain the principles of the invention.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1A illustrates a perspective view of an embodiment of an apparatus for loading an implantable device.

FIG. 1B illustrates a top view of the embodiment of an apparatus for loading an implantable device shown in FIG. 1A.

FIG. 1C illustrates a front view of the embodiment of an apparatus for loading an implantable device shown in FIG. 1A.

FIG. 1D illustrates a back view of the embodiment of an apparatus for loading an implantable device shown in FIG. 1A.

FIG. 2A illustrates a perspective view of another embodiment of an apparatus for loading an implantable device.

FIG. 2B illustrates a front view of the embodiment of an apparatus for loading an implantable device shown in FIG. 2A.

FIG. 2C illustrates a back view of the embodiment of an apparatus for loading an implantable device shown in FIG. 2A.

FIG. 3 illustrates a front view of a further embodiment of an apparatus for loading an implantable device.

FIG. 4A illustrates a front view of a still further embodiment of an apparatus for loading an implantable device.

FIG. 4B illustrates a right side view of the embodiment of an apparatus for loading an implantable device shown in FIG. 4A.

FIG. 4C illustrates a back view of the embodiment of an apparatus for loading an implantable device shown in FIG. 4A.

FIG. 5A illustrates a front view of another embodiment of an apparatus for loading an implantable device.

FIG. 5B illustrates a back view of the embodiment of an apparatus for loading an implantable device shown in FIG. 5A.

FIG. 6 illustrates a perspective view of yet another embodiment of an apparatus for loading an implantable device.

It should be noted that the figures are not drawn to scale and that elements of similar structures or functions are generally represented by like reference numerals for illustrative purposes throughout the figures. It also should be noted that the figures are only intended to facilitate the description of embodiments of the present invention.

### DETAILED DESCRIPTION

The embodiments described herein extend to methods and apparatus for loading an implantable device upon a needle, mandrel, balloon or other structure for supporting or receiving the implantable device. Some of the apparatuses of the present invention are configured to facilitate loading of an implantable device.

Implantable devices may be coated with various materials. It may be desirable to limit contact with the implantable devices prior to implantation. For example, an implantable device may include a stent. A stent may be coated with a beneficial agent to reduce restenosis upon implantation of the device. Handling of a coated stent may affect the efficacy of the coating. A coating may be fragile, thus making a minimization of contact with the implantable device a potential desirable trait. In addition, other implantable devices may themselves be fragile. Furthermore, a loading device may reduce the relevant handling time by providing consistent and/or steady positioning of the implantable device.

These results, whether individually or collectively, can be achieved, according to one embodiment of the present invention, by employing methods, systems, and/or apparatus as shown in the figures and described in detail below.

Turning now to the drawings, FIGS. IA-ID illustrate an embodiment of an apparatus 110 for loading an implantable device 102 an implantable device carrying apparatus 100, such as a needle, mandrel, balloon, and/or other delivery device or stucture. As illustrated, the loading apparatus 110 receives the implantable device 102 that has a lumen 104 that extends from a distal end 106 toward a proximal end 108. This illustrative implantable device 102, in the presently illustrated embodiment, may be a stent. In other embodiments, other implantable devices may be used. For example, and not by way of limitation, a tissue closure element or other implantable device may be used.

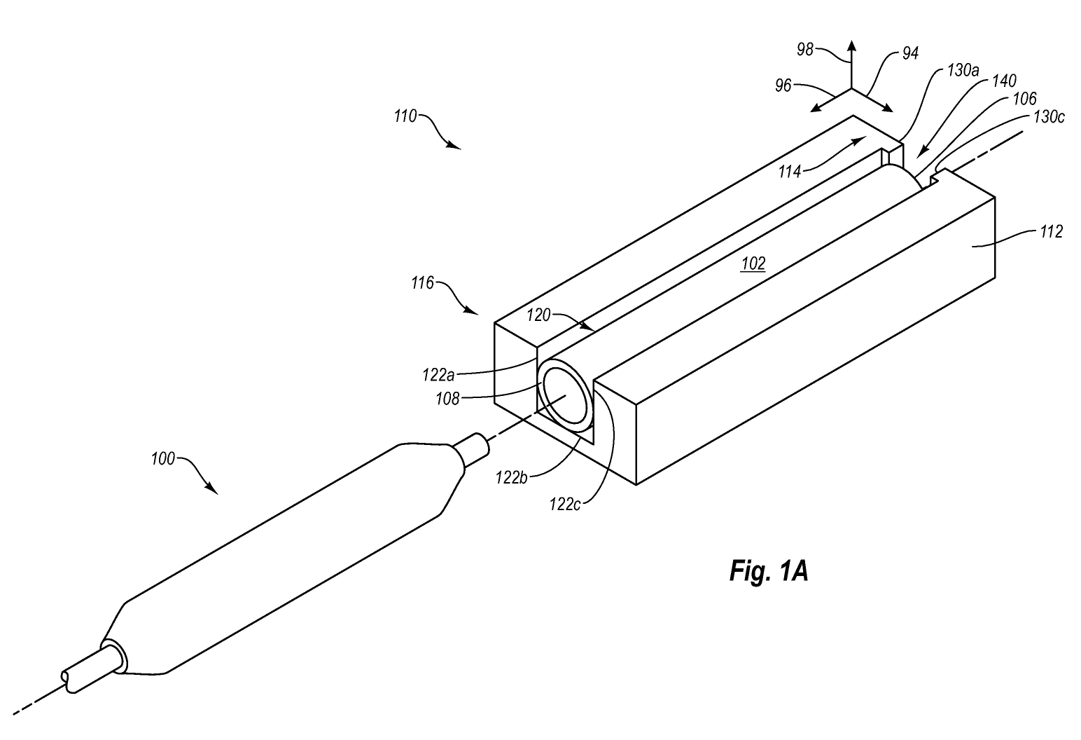

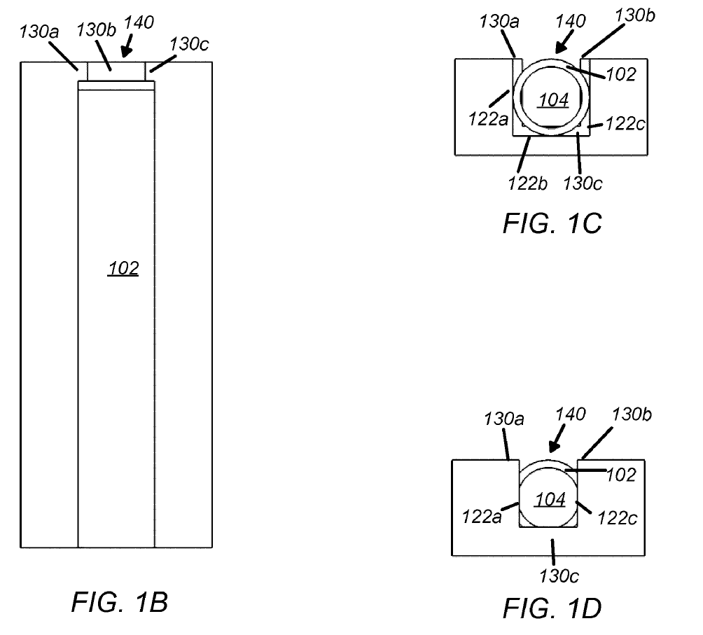

The loading apparatus 110 illustrated in FIG. 1A may include a body 112 having a distal end 114, a proximal end 116, and a channel or support portion 120 extending from the proximal end 116 toward the distal end 114. The body 112 can also include a stop portion 130 and/or a passage 140. The support portion 120 may be configured to receive the implantable device 102 and may include a support surface upon which an implantable device may be supported to generally impede motion in one or more directions. As illustrated in FIG. 1A, the support portion 120 can prohibit or limit motion of the implantable device 102 in a negative vertical direction 98. In the presently illustrated embodiment of FIGS. 1A-1B, the support portion 120 may include three support surfaces 122a, 122b, 122c. The support surfaces 122a, 122b, 122c may be generally planar. In other embodiments, however, the support surfaces may take other shapes or configurations, such as, but not limited to, curved, ridged, and/or other surface shapes or configurations that can be used to retain the implantable device 102 in preparation for loading upon implantable device carrying apparatus 100.

In the present embodiment, two of the support surfaces 122a, 122c may impede the motion of the implantable device 102 in both a positive and a negative lateral direction 94. In other embodiments, a support surface, such as support surface 122b, may not impede the motion of the implantable device 102 in either the positive or the negative lateral directions 94 (i.e. a support surface that is generally parallel to the surface upon which the loading apparatus 110 rests). In further embodiments, only one support surface may impede motion of the implantable device 102 in at least one lateral direction 94. For example, in an embodiment including only support surface 122a and support surface 122b, support surface 122a would impede motion in the negative lateral direction 94 (i.e. toward the left side of the loading apparatus 110 with respect to the orientation shown in FIG. 1C).

The support portion 120 may be sized and/or shaped to generally support an implantable device 102. Supporting an implantable device 102 may include impeding motion in a negative vertical direction 98. The support portion 120 may be approximately the same size as the implantable device 102 or may be larger or smaller than the implantable device 102. For example, a support portion that is approximately half of the length in the longitudinal direction of the implantable device 102 may be used. In still another configuration, the support portion can be approximately half of the height of the implantable device 102. Combinations of these are also possible. Generally, a support portion of any size or shape that may support an implantable device 102 may be used.

Referring to FIGS. 1C and 1D, the loading apparatus 110 may include three stop portions 130a, 130b, 130c. In other embodiments, the loading apparatus 110 may include more or fewer stop portions. The stop portions 130a, 130b, 130c in FIGS. 1A-1D may be located at the distal end 114 of the loading apparatus 110. Alternatively, the stop portions may be located at other locations of the loading apparatus 110, such as at a proximal end 114 of the loading apparatus 110 and/or at one or more locations between the distal end 114 and the proximal end 116.

The stop portions 130a, 130b, 130c may be configured to limit motion of the implantable device 102 in a positive and/or a negative longitudinal direction 96. In the present embodiment, the stop portions 130a, 130b, 130c may limit motion of the implantable device 102 in the negative longitudinal direction 96 or in a direction from the proximal end 116 toward the distal end 114. In other embodiments, additional stop portions may be used to limit motion in one or both of the positive and the negative longitudinal directions 96. For instance, the loading apparatus 110 or the body 112 can include stop portions at either or both the distal end 114 and the proximal end 116.

The stop portions 130a, 130b, 130c of the loading apparatus 110 may contact the implantable device 102 in more than one location. As illustrated in FIG. 1C, each of the stop portions 130a, 130b, 130c may contact different portions of the distal end 114 of the implantable device 102. It will be understood, however, that differing the stop portion location can result in the stop portions contacting or engaging the distal end 114, the proximal end 116, and/or one or more locations between the distal end 114 and the proximal end 116.

Turning now to FIGS. 1A and 1B, formed at the distal end 114 of the body 112 is the passage 140. The passage 140, in the illustrated configuration, is formed from the combination of the stop portions 130a, 130b, 130c and is generally aligned with the lumen 104 of the implantable device 102 when the implantable device 102 is received within the channel or support portion 120. The passage 140 provides an outlet for a portion of the implantable device carrying apparatus 100 that supports or receives the implantable device 102 when is placed within the lumen 104 of the implantable device 102. For example, when inserted within the proximal end 116 of the implantable device 102, a portion of the implantable device carrying apparatus 100 can extend through the passage 140 to aid with locating the implantable device 102 at the desired location of the implantable device carrying apparatus 100. In those instances where the implantable device carrying apparatus 100 does not extend beyond the distal end 114 of the implantable device 102, it will be understood that the loading apparatus 110 or body 112 need not include the passage 140.

As mentioned above, the loading apparatus 110 may be used to load the implantable device 102 onto the implantable device carrying apparatus 100. The following is an example of an embodiment of a method of using the loading apparatus 110 to load the implantable device 102 onto an implantable device carrying apparatus 100.

An implantable device 102 may be positioned with respect to the loading apparatus 110. The implantable device carrying apparatus 100 may be inserted into the lumen 104 of the implantable device 102 via the proximal end 116. Inserting the implantable device carrying apparatus 100 into the lumen 104 of the implantable device 102 may cause the implantable device 102 to move in a negative longitudinal direction 96 or toward the distal end 112 of the loading apparatus 110. In embodiments where the loading apparatus 110 includes a stop portion, the stop portion may limit this negative longitudinal motion of the loading apparatus 110, such that the implantable device carrying apparatus 100 may be inserted to a desired position relative to the implantable device 102 and optionally extend through all or a portion of the passage 140. The implantable device carrying apparatus 100 and the implantable device 102 may then be removed from the loading apparatus 110.

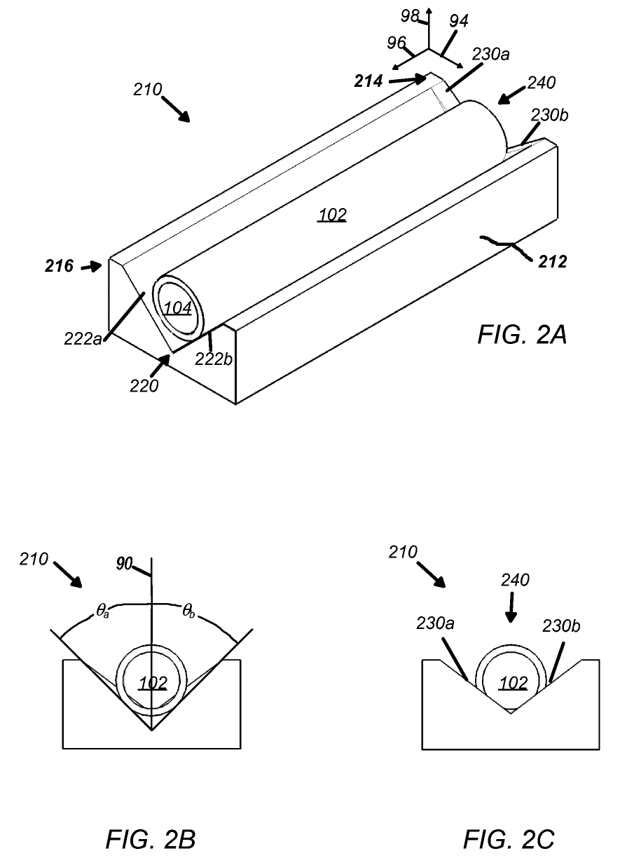

FIGS. 2A-2C illustrate another embodiment of an apparatus 210 for loading an implantable device 102. As illustrated, the implantable device 102 may include a stent. The loading apparatus 210 may include a body 212 having a distal end 214, a proximal end 216, and a channel or support portion 220. The body 212 can also include a stop portion 230 and/or a passage 240. The support portion 220 may be configured to receive the implantable device 102 and may include two support surfaces 222a, 222b. Support surfaces 222a, 222b may generally impede the motion of the implantable device 102 in both a positive and a negative lateral direction 94. As illustrated in FIG. 2A, the support portion 120 can prohibit or limit motion of the implantable device 102 in and in the negative vertical direction 98.

Support surfaces 222a, 222b may be oriented at an angle θₐ, θ_{b} with respect to a plane 90 defined by the longitudinal direction 96 and the vertical direction 98 (Shown in FIG. 2B). Orienting support surfaces 222a, 222b non-orthogonally with respect to the plane 90 may generally impede motion in both the lateral direction 94 and the vertical direction 98. The closer the support surfaces 222a, 222b may be to orthogonal with respect to the plane 90, generally the more the support surfaces 222a, 222b may impede motion in the lateral direction 94.

The support portion 220 may be sized and/or shaped to generally support an implantable device 102. The support portion 220 may be approximately the same length as the implantable device 102. However, the support surfaces 222a, 222b may not generally surround the implantable device 102 on three sides in comparison with the exemplary support surfaces 122a, 122b, 122c shown in connection with the previous embodiment.

Referring to FIGS. 2B and 2C, the loading apparatus 210 may include two stop portions 230a, 230b. The stop portions 230a, 230b in FIGS. 2A-2C may be located at the distal end 214 (shown in FIG. 2A) of the loading apparatus 210. The stop portions 230a, 230b may be configured to limit motion of the implantable device 102 in the negative longitudinal direction 96. The stop portions 230a, 230b may contact the implantable device 102 in more than one location.

Turning now to FIGS. 2A and 2B, formed at the distal end 214 of the body 212 is the passage 240. The passage 240 may be defined by the area above stop portions 230a, 230b and is generally aligned with the lumen 104 of the implantable device 102. A user using the loading apparatus 210 of the present embodiment may be able to access the lumen 104 while the implantable device 102 is being supported by the support portion 220 and support surfaces 222a, 222b.

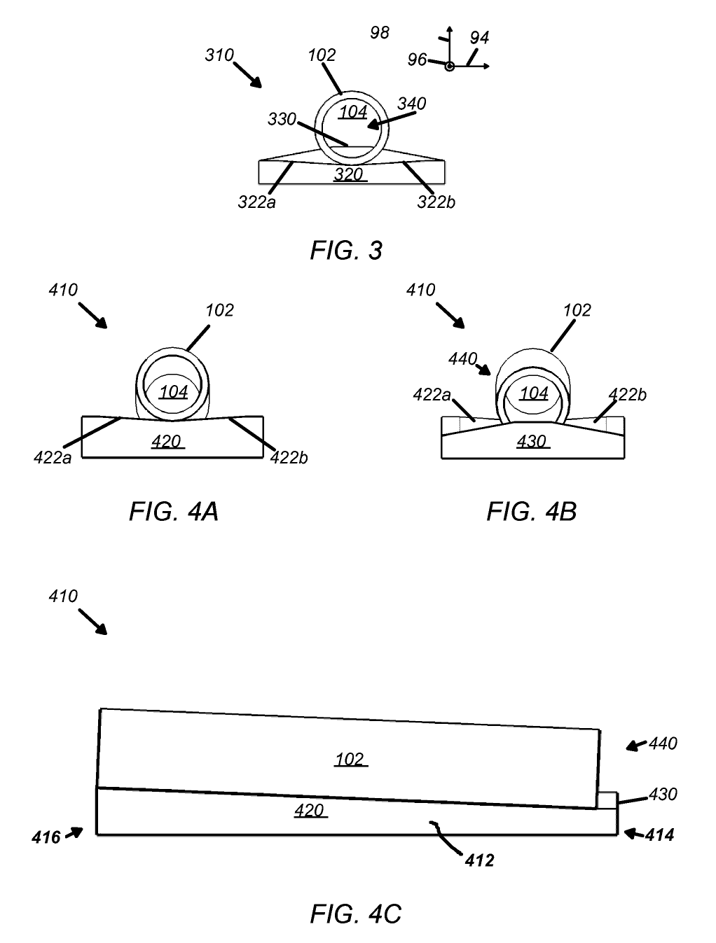

FIG. 3 illustrates a front view of a further embodiment of an apparatus 310 for loading an implantable device 102. The loading apparatus 310 of this further embodiment may be functionally similar to that of the loading apparatus 210 previously described above and shown in FIGS. 2A-2C in most respects, wherein certain features will not be described in relation to the further embodiment wherein those components may function in the manner as described above and are hereby incorporated into the alternative embodiment described below.

Support surfaces 322a, 322b may be oriented at an angle (not shown) with respect to a plane (not shown) defined by the longitudinal direction 96 and the vertical direction 98. The supports surfaces 322a, 322b may be oriented at a less acute angle with respect to the plane defined by the longitudinal direction 96 and the vertical direction 98 in comparison to the support surfaces 222a, 222b shown in FIG. 2B. Additionally, in contrast to the previous embodiment, the loading apparatus 310 of the present embodiment may include a single stop portion 330. The use of a single stop portion 330 may create a larger passage 340, while still limiting motion in the negative longitudinal direction 96.

FIGS. 4A-4C illustrate a still further embodiment of an apparatus 410 for loading an implantable device 102. The loading apparatus 410 may be functionally similar to that of the loading apparatus 310 previously described above and shown in FIG. 3 in most respects, wherein certain features will not be described in relation to the further embodiment wherein those components may function in the manner as described above and are hereby incorporated into the alternative embodiment described below.

As illustrated in FIGS. 4A-4C, the loading apparatus 410 may include a body 412 having a distal end 414, a proximal end 416, and a channel or support portion 420. The body 412 can also include a stop portion 430 and/or a passage 440. The support portion 420 may be sloped relative to the stop portion 430. The support portion 420 may be sloped relative to the stop portion 430 from a proximal end 414 of the loading apparatus 410 toward a distal end 412 of the loading apparatus 410. Sloping the support portion 420 may facilitate loading the implantable device 102 onto an implantable device carrying apparatus 100 (Shown in FIG. 1A) by generally guiding the implantable device 102 toward the stop portion 430.

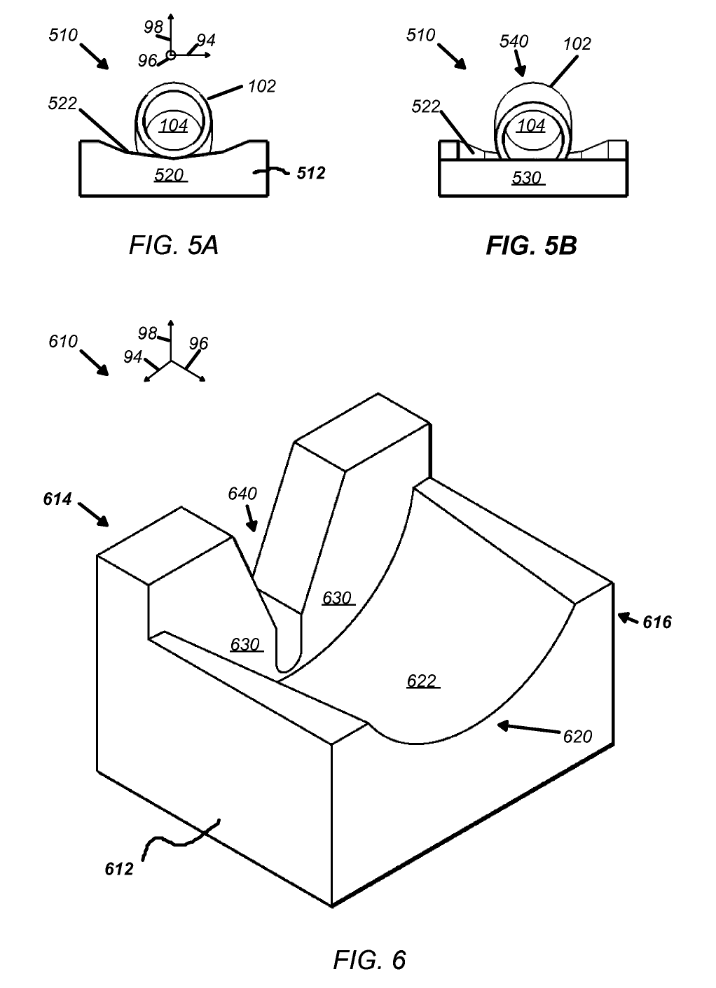

FIGS. 5A-5B illustrate another embodiment of an apparatus 510 for loading an implantable device 102. The loading apparatus 510 may be functionally similar to that of the loading apparatus 410 previously described above and shown in FIGS. 4A-4C in most respects, wherein certain features will not be described in relation to the further embodiment wherein those components may function in the manner as described above and are hereby incorporated into the alternative embodiment described below.

As illustrated in FIGS. 5A-5B, the loading apparatus 510 may include a body 512 having a support portion 520, a stop portion 530, and/or a passage 540. The support portion 520 may include a support surface 522, which may be curved. The support surface 522 may be concave toward the positive vertical direction 98.

The support surface 522 may generally impede the motion of the implantable device 102 in both a positive and a negative lateral direction 94. As illustrated in FIG. 5B, the support surface 522 may impede the motion of the implantable device 102 in the negative vertical direction 98.

Support surface 522 may be generally defined by a radius (not shown). Using a curved support surface 522 may impede motion in both the lateral direction 94 and the vertical direction 98. Typically, the smaller the radius with respect to the size of the implantable device 102, the more the support surface 522 may impede motion in the lateral direction 94.

The stop portion 530, in the present embodiment, may include a top surface (not shown) that may be orthogonal to a plane (not shown) defined generally by the lateral and longitudinal axes 94, 96.

FIG. 6 illustrates a perspective view of an embodiment of an apparatus 610 for loading an implantable device (not shown). The loading apparatus 610 may include a body 612 having a distal end 414, a proximal end 416, and a support portion 620. The body 612 can also include a stop portion 630 and/or a passage 640. The support portion 620 may be configured to receive an implantable device.

The support portion 620 may be sloped relative to the stop portion 630 from a proximal end 616 of the loading apparatus 610 toward a distal end 614 of the loading apparatus 610. Sloping the support portion 620 may facilitate loading an implantable device onto an implantable device carrying apparatus (not shown) by generally guiding the implantable device toward the stop portion 630.

The support portion 620 may include one support surface 622. The support surface 622 may be generally curved and may be concave toward the positive vertical direction 98.

The support surface 622 may generally impede the motion of the implantable device in both a positive and a negative lateral direction 94. As illustrated in FIG. 6, support surface 622 may impede the motion of the implantable device 102 in the negative vertical direction 98.

Support surface 622 may be generally defined by a radius (not shown). Using a curved support surface 622 may impede motion in both the lateral direction 94 and the vertical direction 98. Typically, the smaller the radius with respect to the size of the implantable device, the more the support surface 622 may impede motion in the lateral direction 94.

The loading apparatus 610 may include a stop portion 630. The stop portion 630, in the present embodiment, may be located at a distal end 614 of the loading apparatus 610. The stop portion 630 may limit motion of the implantable device in the negative longitudinal direction 96 and/or may contact a portion of the implantable device.

The passage 640 may be formed at the distal end 614 of the body 612. The passage 640, in the illustrated configuration, is formed from the stop portion 630 and may be generally aligned with the lumen (not shown) of the implantable device when the implantable device is received within the channel or support portion 620. The passage 640 provides an outlet for a portion of the implantable device carrying apparatus (not shown) that supports or receives the implantable device when is placed within the lumen of the implantable device. For example, when inserted within the proximal end of the implantable device, a portion of the implantable device carrying apparatus can extend through the passage 640 to aid with locating the implantable device at the desired location of the implantable device carrying apparatus. In those instances where the implantable device carrying apparatus does not extend beyond the distal end of the implantable device, it will be understood that the loading apparatus 610 or body 612 need not include the passage 640

The passage 640 may include a generally Y-shaped opening. A Y-shaped opening may facilitate removal of the implantable device and an implantable device carrying apparatus.

The support surface 622, in the present embodiment, may support multiple implantable devices. For example, the embodiments of loading apparatus 110, 210, 310, 410, 510 shown in FIGS. 1A-5B have been generally sized to support one implantable device. The loading apparatus 610 may support multiple implantable devices. This may facilitate faster loading of multiple implantable devices because, rather than individually positioning and then loading each implantable device, multiple implantable devices may be positioned as a group and then individually loaded onto an implantable device carrying apparatus.

As described in connection with FIGS. 1A-1D, the loading apparatus 110, 210, 310, 410, 510, 610 may be used to load an implantable device 102 onto an implantable device carrying apparatus (not shown). Various features disclosed with respect to the various embodiments may provide certain advantages that may be generally combined to achieve desired results.

The invention is susceptible to various modifications and alternative means, and specific examples thereof have been shown by way of example in the drawings and are herein described in detail. It should be understood, however, that the invention is not to be limited to the particular devices or methods disclosed, but to the contrary, the invention is to cover all modifications, equivalents, and alternatives falling within the spirit and scope of the claims.

What is claimed is:

1. An apparatus for loading an implantable device, comprising:
a support portion configured to receive the implantable device; and
a stop portion configured to limit longitudinal motion of the implantable device in at least one direction.

2. The apparatus of claim 1, said support portion including at least one support surface.

3. The apparatus of claim 2, said at least one support surface being configured to impede lateral motion of the implantable device in at least one direction.

4. The apparatus of claim 2, said at least one support surface being concave.

5. The apparatus of claim 2, said at least one support surface being curved.

6. The apparatus of claim 2, wherein a first support surface and a second support surface intersect in an acute angle.

7. The apparatus of claim 1, said stop portion including a passage configured to allow access to a lumen of the implantable device.

8. The apparatus of claim 1, said support portion being sloped relative to said stop portion from a proximal end of said support portion toward a distal end of said support portion.

9. An apparatus for loading an implantable device, comprising:
a support portion configured to receive the implantable device, said support portion including at least one support surface; and
a stop portion configured to limit longitudinal motion of the implantable device in at least one direction, said stop portion including a passage configured to allow access to a lumen of the implantable device.

10. The apparatus of claim 9, said at least one support surface being configured to impede lateral motion of the implantable device in at least one direction.

11. The apparatus of claim 9, said at least one support surface being concave.

12. The apparatus of claim 9, said at least one support surface being curved.

13. The apparatus of claim 9, wherein a first support surface and a second support surface intersect in an acute angle.

14. The apparatus of claim 9, said support portion being sloped relative to said stop portion from a proximal end of said support portion toward a distal end of said support portion.

15. An apparatus for loading an implantable device, comprising:
a support portion configured to receive the implantable device, said support portion including at least one support surface, said at least one support surface being configured to impede lateral motion of the implantable device in at least one direction; and
a stop portion configured to limit longitudinal motion of the implantable device in at least one direction, said stop portion including a passage configured to allow access to a lumen of the implantable device;
said support portion being sloped relative to said stop portion from a proximal end of said support portion toward a distal end of said support portion;

16. The apparatus of claim 15, said at least one support surface being concave.

17. The apparatus of claim 15, said at least one support surface being curved.

18. The apparatus of claim 15, wherein a first support surface and a second support surface intersect in an acute angle.

19. A method for loading an implantable device, comprising:
positioning an implantable device within a loading apparatus, the loading apparatus comprising:
a support portion configured to receive the implantable device and the support portion being configured to impede lateral motion of the implantable device in at least one direction; and
a stop portion configured to limit longitudinal motion of the implantable device in at least one direction;
inserting an implantable device carrying apparatus into the implantable device; and
removing the implantable device carrying apparatus and the implantable device from the loading apparatus.

20. The method of claim 19, further comprising positioning multiple implantable devices within the loading apparatus.
